(19) United States
(12) Patent Application Publication
(10) Pub. No.: US 2025/0108216 A1
(43) Pub. Date: Apr. 3, 2025

(54) Applying Tumor Treating Fields (TTFields) with Temperature Sensors Positioned Between the Electrode Elements and the Subject's Skin

(71) Applicant: Novocure GmbH, Root D4 (CH)

(72) Inventor: Yoram WASSERMAN, Haifa (IL)

(73) Assignee: Novocure GmbH, Root D4 (CH)

(21) Appl. No.: 18/897,223

(22) Filed: Sep. 26, 2024

Related U.S. Application Data
(60) Provisional application No. 63/541,351, filed on Sep. 29, 2023.

(51) Int. Cl. A61N 1/36; A61N 1/04

(52) U.S. Cl. CPC A61N 1/36031; A61N 1/0476; A61N 1/0496; A61N 1/36034

(57) ABSTRACT

Alternating electric fields (e.g., tumor treating fields or TTFields) can be applied to a subject's body by positioning electrode elements on opposite sides of a target region in the subject's body, and applying an AC voltage between the opposing electrode elements. Temperature sensors that are positioned in front of the electrode elements (i.e., between the electrode elements and the subject's skin) and in thermal contact with the subject's skin are used to monitor the temperature of the subject's skin. The current that is applied to the electrode elements is adjusted to the highest level possible that will not cause the subject's skin to exceed the safety threshold (e.g., 41° C.). Optionally, one or more heat exchangers is included to remove heat from the electrode elements, in which case it will be possible to drive larger currents through the electrode elements without exceeding the safety threshold.

### CROSS REFERENCE TO RELATED APPLICATIONS

This Application claims the benefit of U.S. Provisional Application 63/541,351, filed Sep. 29, 2023, which is incorporated herein by reference in its entirety.

### BACKGROUND

Tumor Treating Fields (TTFields) therapy is a proven approach for treating tumors using alternating electric fields at frequencies e.g., between 50 kHz-5 MHz, more commonly 100-500 KHz. The alternating electric fields are induced by electrode assemblies (e.g., arrays of capacitively coupled electrodes, also called transducer arrays) placed on the subject's skin on opposite sides of the subject's body. When an AC voltage is applied between opposing electrode assemblies, an AC current is coupled through the electrode assemblies and into the subject's body.

Higher currents are strongly correlated with higher efficacy of treatment. But the current cannot be set arbitrarily high because both the electrode assemblies and the skin beneath the electrode assemblies heat up during use, and safety considerations require the skin temperature not to exceed a threshold value (e.g., 41° C.).

One example of an electrode assembly that can be used to apply alternating electric fields to a subject's body is described in U.S. Pat. No. 8,715,203. In this example, thermistors are positioned in small holes in the center of the electrode elements within the electrode assemblies, in thermal contact with the electrode elements. Another example of an electrode assembly that can be used to apply alternating electric fields to a subject's body is described in Pub. No. US 2021/0402179. In this example, thermistors are positioned behind the electrode elements, in thermal contact with the electrode elements. The prior art systems rely on signals from these thermistors to ensure that the current is low enough to prevent the temperature of the subject's skin from exceeding the safety threshold.

### SUMMARY OF THE INVENTION

One aspect of the invention is directed to a first apparatus for applying an alternating electric field to a subject's body. The first apparatus comprises at least one electrode element, a support, and a plurality of temperature sensors. Each electrode element has a respective front face that has a respective area. The support is configured to hold the at least one electrode element adjacent to the subject's body, so that the front faces of the at least one electrode element face the subject's body. The plurality of temperature sensors are positioned in front of the at least one electrode element so that when the support holds the at least one electrode element adjacent to the subject's body, the plurality of temperature sensors will be situated between the front faces of the at least one electrode element and the subject's body, in thermal contact with the subject's body.

In some embodiments of the first apparatus, each of the temperature sensors comprises a thermistor.

Some embodiments of the first apparatus further comprise a plurality of insulated wires positioned in front of the at least one electrode element, wherein the plurality of insulated wires is configured to route electrical signals to the temperature sensors. Optionally, in these embodiments, the plurality of temperature sensors and the plurality of insulated wires collectively occupy an area that is less than 10% of a sum of all the areas of all the electrode elements.

Some embodiments of the first apparatus further comprise a flex circuit configured to support the plurality of temperature sensors and to provide an electrical interface with the plurality of temperature sensors. Optionally, in these embodiments, the flex circuit has an insulating substrate and a plurality of conductive traces, the flex circuit is oriented so that the insulating substrate is in front of the plurality of conductive traces, and the insulating substrate is shaped and dimensioned to prevent the plurality of conductive traces from making contact with the subject's body.

In some embodiments of the first apparatus, the support is positioned behind the at least one electrode element.

Some embodiments of the first apparatus further comprise a heat exchanger disposed in thermal contact with the at least one electrode element, wherein the heat exchanger is positioned behind the at least one electrode element and is configured to remove heat from the at least one electrode element.

Optionally, in the embodiments described in the previous paragraph, the heat exchanger can rely on a flow of a liquid to carry the heat away. Optionally, in the embodiments described in the previous paragraph, the heat exchanger can comprise a passive heat sink positioned in contact with something cold.

Some embodiments of the first apparatus further comprise a layer of insulating material having a dielectric constant greater than 10 disposed on the front faces of the at least one electrode element. In these embodiments, the plurality of temperature sensors are positioned in front of the layer of insulating material.

Some embodiments of the first apparatus further comprise a layer of insulating material having a dielectric constant greater than 10 disposed on the front faces of the at least one electrode element, wherein the layer of insulating material has a front face; and a sheet of graphite disposed on the front face of the layer of insulating material. In these embodiments, the plurality of temperature sensors are positioned in front of the layer of graphite.

Optionally, the embodiments described in the previous paragraph can further comprise a heat exchanger disposed in thermal contact with the at least one electrode element, wherein the heat exchanger is positioned behind the at least one electrode element and is configured to remove heat from the at least one electrode element.

Some embodiments of the first apparatus further comprise a sheet of graphite and a layer of conductive adhesive. The sheet of graphite is disposed in front of the plurality of temperature sensors so that when the support holds the at least one electrode element adjacent to the subject's body, the sheet of graphite will be situated between the plurality of temperature sensors and the subject's body. And the layer of conductive adhesive is disposed in front of the sheet of graphite so that when the support holds the at least one electrode element adjacent to the subject's body, the layer of conductive adhesive will be situated between the sheet of graphite and the subject's body.

Another aspect of the invention is directed to a second apparatus for applying an alternating electric field to a subject's body. The second apparatus comprises a plurality of electrode elements, a support, a flex circuit, and a plurality of temperature sensors. Each of the electrode elements has a respective front face that has a respective area. The support is configured to hold the plurality of electrode elements adjacent to the subject's body, so that the front faces of the plurality of electrode elements face the subject's body. The flex circuit has an insulating substrate and a plurality of conductive traces, and the flex circuit is positioned so that when the support holds the plurality of electrode elements adjacent to the subject's body, the flex circuit will be situated between the front faces of the plurality of electrode elements and the subject's body. The plurality of temperature sensors are affixed to the flex circuit, and the plurality of conductive traces of the flex circuit are configured to provide an electrical interface with the plurality of temperature sensors.

In some embodiments of the second apparatus, each of the temperature sensors comprises a thermistor.

In some embodiments of the second apparatus, the flex circuit is shaped and dimensioned to cover less than 10% of a sum of all the areas of all the electrode elements. Optionally, in these embodiments, a sum of all the areas of all the electrode elements is at least 10 cm².

In some embodiments of the second apparatus, the flex circuit is oriented so that the insulating substrate is in front of the plurality of conductive traces, and the insulating substrate is shaped and dimensioned to prevent the plurality of conductive traces from making contact with the subject's body.

Some embodiments of the second apparatus further comprise a heat exchanger disposed in thermal contact with the plurality of electrode elements, wherein the heat exchanger is positioned behind the plurality of electrode elements and is configured to remove heat from the plurality of electrode elements.

Another aspect of the invention is directed to a third apparatus for applying an alternating electric field to a subject's body. The third apparatus comprises at least one electrode element, a support, a plurality of temperature sensors, and a flex circuit. Each electrode element has a respective front face that has a respective area. The support is configured to hold the at least one electrode element adjacent to the subject's body, so that the front faces of the at least one electrode element face the subject's body. The plurality of temperature sensors are positioned in front of the at least one electrode element so that when the support holds the at least one electrode element adjacent to the subject's body, the plurality of temperature sensors will be situated between the front faces of the at least one electrode element and the subject's body, in thermal contact with the subject's body. The flex circuit has an insulating substrate and a plurality of conductive traces. The at least one electrode element comprises a plurality of electrode elements. The flex circuit is positioned so that when the support holds the plurality of electrode elements adjacent to the subject's body, the flex circuit will be situated between the front faces of the plurality of electrode elements and the subject's body. The plurality of temperature sensors are affixed to the flex circuit, and the plurality of conductive traces of the flex circuit are configured to provide an electrical interface with the plurality of temperature sensors.

In some embodiments of the third apparatus, each of the temperature sensors comprises a thermistor.

In some embodiments of the third apparatus, the flex circuit is shaped and dimensioned to cover less than 10% of a sum of all the areas of all the electrode elements. Optionally, in these embodiments, a sum of all the areas of all the electrode elements is at least 10 cm².

Another aspect of the invention is directed to a first method for applying an alternating electric field to a subject's body. The first method comprises positioning at least one first electrode element against the subject's body, with a plurality of first temperature sensors sandwiched between the at least one first electrode element and the subject's body so that the first temperature sensors are in thermal contact with the subject's body, wherein each of the at least one first electrode elements has a respective rear surface. The first method also comprises positioning a first heat exchanger in thermal contact with the rear surfaces of the at least one first electrode element. The first method also comprises positioning at least one second electrode element against the subject's body, with a plurality of second temperature sensors sandwiched between the at least one second electrode element and the subject's body so that the second temperature sensors are in thermal contact with the subject's body, wherein each of the at least one second electrode elements has a respective rear surface. The first method also comprises positioning a second heat exchanger in thermal contact with the rear surfaces of the at least one second electrode element. The first method also comprises applying an alternating voltage between the at least one first electrode element and the at least one second electrode element; passing a first cooling fluid through the first heat exchanger to remove heat from the at least one first electrode element; passing a second cooling fluid through the second heat exchanger to remove heat from the at least one second electrode element; determining a plurality of first temperatures based on outputs of the plurality of first temperature sensors; and determining a plurality of second temperatures based on outputs of the plurality of second temperature sensors. The first method also comprises adjusting at least one of (a) an amplitude of the alternating voltage and (b) the passing of the first cooling fluid through the first heat exchanger based on the determined plurality of first temperatures; and adjusting at least one of (i) an amplitude of the alternating voltage and (ii) the passing of the second cooling fluid through the second heat exchanger based on the determined plurality of second temperatures.

In some instances of the first method, the at least one first electrode element comprises a plurality of first electrode elements, and each of the plurality of first temperature sensors is sandwiched between a respective one of the plurality of first electrode elements and the subject's body. In these instances, the at least one second electrode element comprises a plurality of second electrode elements, and each of the plurality of second temperature sensors is sandwiched between a respective one of the plurality of second electrode elements and the subject's body.

In some instances of the first method, each of the first temperature sensors and each of the second temperature sensors comprises a thermistor.

Another aspect of the invention is directed to a kit for applying an alternating electric field to a subject's body. The kit comprises (1) a first component that includes (a) a plurality of electrode elements, wherein each of the electrode elements has a respective front face that has a respective area, and (b) a support configured to hold the plurality of electrode elements adjacent to the subject's body, so that the front faces of the plurality of electrode elements face the subject's body; and (2) a second component that includes (i) a flex circuit having an insulating substrate and a plurality of conductive traces, and (ii) a plurality of temperature sensors affixed to the flex circuit. The plurality of conductive traces of the flex circuit are configured to provide an electrical interface with the plurality of temperature sensors. And the flex circuit is configured to fit between the front faces of the plurality of electrode elements and the subject's body when the support holds the plurality of electrode elements adjacent to the subject's body.

### BRIEF DESCRIPTION OF THE DRAWINGS

FIG. 1 depicts one example of a system that uses two electrode assemblies to apply TTFields to a subject's body.

FIG. 2 depicts one component that is contained within each of the electrode assemblies depicted in FIG. 1.

FIG. 3 depicts a second component that is contained within each of the electrode assemblies depicted in FIG. 1.

FIG. 4 depicts the component depicted in FIG. 2 affixed to the component depicted in FIG. 3.

Various embodiments are described in detail below with reference to the accompanying drawings, wherein like reference numerals represent like elements.

### DESCRIPTION OF THE PREFERRED EMBODIMENTS

Notably, the thermistors in the prior art systems are positioned in thermal contact with electrode elements within the electrode assemblies (as opposed to being in direct thermal contact with the subject's skin). As a result, a thermal gradient exists between the thermistors and the subject's skin. But because this thermal gradient is relatively small, the temperature measurements obtained at the electrode elements can still be used as a reliable surrogate for the temperature of the subject's skin, as long as the system accounts for the thermal gradient. The prior art systems account for this thermal gradient by controlling the current so that the temperature of the thermistors does not exceed a threshold value that is lower than the safety threshold. For example, since the safety threshold is 41° C., some prior art systems control the current to the electrode assemblies so that the temperature of the thermistors remains below 39.7° C. And because the thermal gradient between the thermistors and the subject's skin is relatively small, keeping the electrode elements' temperature below 39.7° C. (using thermistor-based measurements) is sufficient to ensure that the skin temperature remains below 41° C.

One approach for increasing the current (and thereby increasing the efficacy of treatment) without exceeding the safety threshold of 41° C. is to cool the electrode assemblies (e.g., using a heat exchanger). Examples of suitable heat exchangers for this purpose include those that rely on ice packs or circulating cool water, etc. But in systems that use higher currents coupled with a heat exchanger, the temperature gradient between the subject's skin and thermistors positioned in thermal contact with the electrode elements can increase to the point where measuring the temperatures of the electrode elements no longer provides a reliable surrogate for direct measurement of the temperature at the subject's skin. This is especially true when the thermistors are positioned behind the electrode assemblies, and the heat exchanger is also positioned behind the electrode assemblies. For with this arrangement of components, the most cooling will be applied to the thermistors, a lower level of cooling will be applied to the electrode elements, and an even lower level of cooling will be applied to the subject's skin. And this can cause a significant discrepancy in temperature between the thermistors and the subject's skin.

The embodiments described below overcome this problem by positioning temperature sensors (e.g., thermistors) in *front* of the electrode elements (i.e., between the electrode elements of the electrode assemblies and the subject's skin), in thermal contact with the subject's body. By positioning the temperature sensors closer to the subject's skin (as compared to the prior art positioning), the temperature gradient between the temperature sensors and the subject's skin is significantly reduced, which makes the temperature readings obtained from the temperature sensors much more reliable.

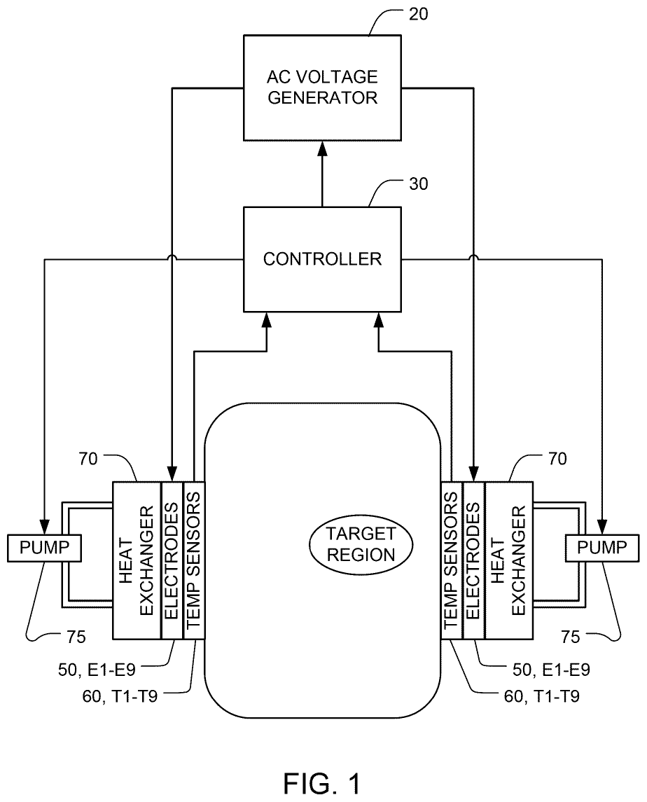

FIG. 1 depicts one example of a system that uses two electrode assemblies 50, 60, E1-E9, T1-T9 to apply TTFields to a subject's body. Each of the electrode assemblies has a set of temperature sensors 60 that are positioned in front of the electrode elements E1-E9 within each of the electrode assemblies. And a separate heat exchanger 70 is optionally used to cool each of the electrode assemblies. In alternative embodiments (not shown), a single heat exchanger could be used to cool both of the electrode assemblies.

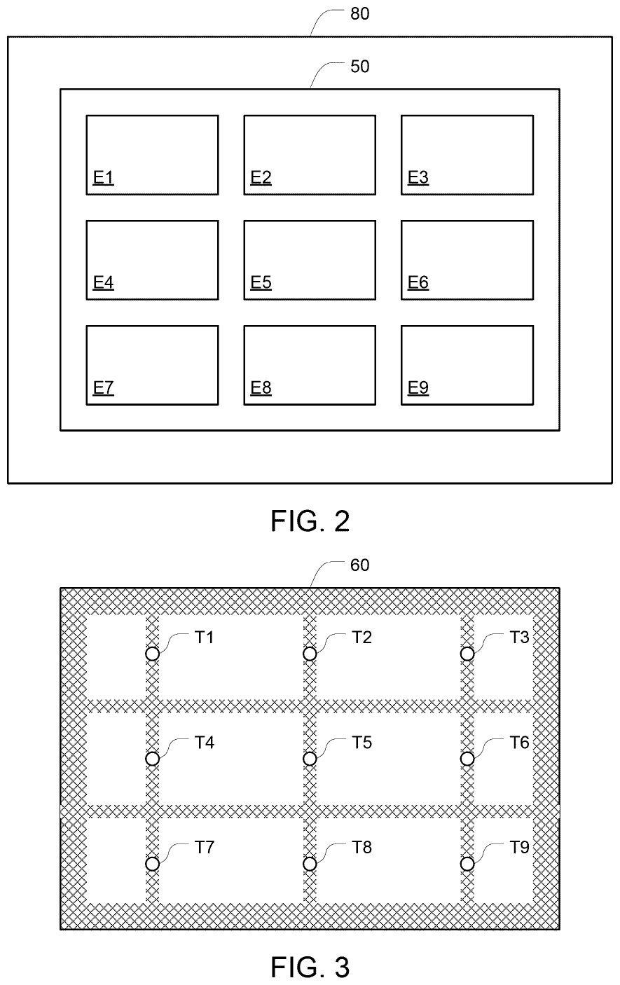

Each of the electrode assemblies in the FIG. 1 embodiment includes at least one electrode element E1-E9, and each of these electrode elements has a respective front face that has a respective area. A support (e.g., the PCB 50 and flexible backing 80) described below in connection with FIG. 2 is configured to hold the at least one electrode element E1-E9 adjacent to the subject's body, so that the front faces of the at least one electrode element face the subject's body. A plurality of temperature sensors T1-T9 (e.g., thermistors) are positioned in front of the at least one electrode element E1-E9 so that when the support holds the at least one electrode element adjacent to the subject's body, the plurality of temperature sensors T1-T9 will be situated between the front faces of the at least one electrode element E1-E9 and the subject's body, in thermal contact with the subject's body.

FIGS. 2-4 depict an example of a suitable two-part approach for implementing the support that holds the electrode elements E1-E9 in each of the electrode assemblies adjacent to the subject's body, and for positioning the temperature sensors T1-T9 between the front faces of the electrode elements E1-E9 and the subject's body.

The first part of the two-part approach is depicted in FIG. 2, which has a first flex circuit 50 (i.e., a flexible PCB) with a plurality of conductive metal pads (e.g., copper pads) provided on the front face thereof. These metal pads serve as the electrode elements E1-E9. The first flex circuit 50 can be implemented, for example, using copper traces and pads that have been formed on a polyimide substrate. But alternative metals and/or alternative insulating substrates may be used in place of the copper and polyimide. A flexible backing 80 is provided behind the substrate of the first flex circuit 50.

Optionally, one or more additional layers of material may be positioned in front of the electrode elements E1-E9 within the first part of the two-part approach. For example, in some embodiments, a layer of an insulating material with a high dielectric constant (e.g., >10, >20, or >40) is positioned on the front face of each of the electrode elements E1-E9. This layer of insulating material can be implemented as a single large sheet that covers all of the electrode elements E1-E9, or as a set of nine smaller pads, each of which is dimensioned and positioned to cover a respective one of the electrode elements E1-E9. The purpose of the layer of insulating material is to capacitively couple the electrode elements E1-E9 to the subject's body via whatever conductive layers are positioned between the layer of insulating material and the subject's body. On the other hand, when a layer of insulating material is not provided, the electrode elements E1-E9 will be conductively coupled to the subject's body via whatever conductive layers are positioned between the electrode elements E1-E9 and the subject's body.

In another example, a thin sheet of graphite or another anisotropic material may be positioned in front of all of the electrode elements E1-E9 within the first part of the two-part approach. The purpose of this layer is to spread the heat and current out in directions that are parallel to the face of the electrode assemblies. In yet another example, a layer of insulating material is positioned on the front face of each of the electrode elements E1-E9 as described above, and a thin sheet of graphite is positioned in front of the layer of insulating material (optionally with a thin sheet of a conductive adhesive positioned therebetween). This example provides both capacitive coupling and the spreading out of heat and current within the first part of the two-part approach.

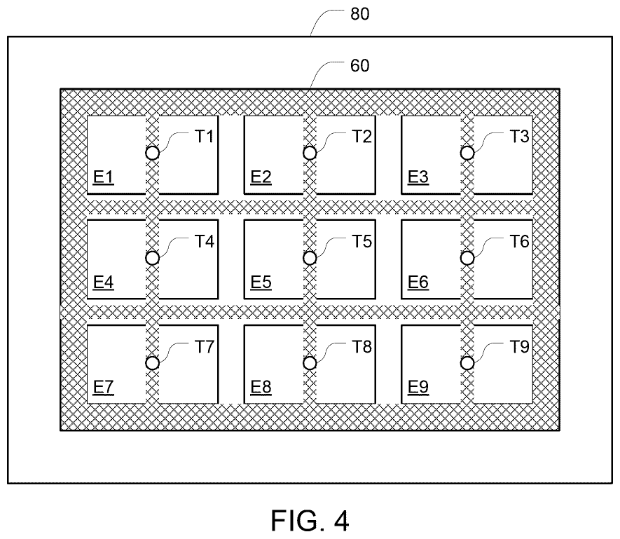

The second part of the two-part approach is depicted in FIG. 3, which has a second flex circuit 60 and a plurality of temperature sensors T1-T9 (e.g., thermistors) affixed thereto. This second flex circuit 60 is configured to support the plurality of temperature sensors T1-T9 and to provide an electrical interface with the plurality of temperature sensors.

The two parts can be assembled by pressing the second flex circuit 60 (of the second part) against the front face of the electrode elements E1-E9 (of the first part), resulting in the configuration depicted in FIG. 4, in which the flexible backing 80 is in the rear, followed by the first flex circuit 50, followed by the second flex circuit 60 at the frontmost position. The electrode elements E1-E9 are disposed on the front face of the first flex circuit 50.

In this example, the second flex circuit 60 is situated between the front faces of the plurality of electrode elements E1-E9 and the subject's body. The second flex circuit has an insulating substrate and a plurality of conductive traces, and the second flex circuit 60 is oriented so that the insulating substrate is in front of the plurality of conductive traces. The insulating substrate is shaped and dimensioned to prevent the plurality of conductive traces on the second flex circuit 60 from making contact with the subject body. This can be accomplished, e.g., by making sure that the conductive traces do not extend all the way to the edges of the insulating substrate. In these embodiments, it is preferable for the flex circuit to be shaped and dimensioned to cover less than 10% of a sum of all the areas of all the electrode elements so that the coupling between the electrode elements E1-E9 and the subject's body is not unduly blocked. In some preferred embodiments, the sum of all the areas of all the electrode elements E1-E9 is at least 10 cm². (These embodiments are preferred because larger areas are advantageous for delivering alternating electric fields and for dissipating heat.)

Optionally, additional layers positioned behind the thermistors T1-T9 can be included in the electrode assembly. For example, a sheet of graphite (not shown) that covers the entire front face of the first flex circuit 50 may be positioned immediately in front of the electrode elements E1-E9 in order to spread the heat and current out in directions that are parallel to the front face of the electrode assembly. Graphite has an additional advantage in that it also prevents ions from flowing into or out of the subject's body. Other materials that block ions and have high thermal conductivity (e.g., dry conductive carbon adhesive) may also be used instead of graphite.

The thermal contact between the temperature sensors T1-T9 and the subject's body can be either direct thermal contact or indirect thermal contact through intervening components. In some preferred embodiments, steps are taken to improve the thermal contact between the temperature sensors T1-T9 and the subject's body to the extent possible. For example, thin layers of conductive adhesive and/or conductive hydrogel (not shown) can be positioned between the temperature sensors T1-T9 and the subject's body. Alternatively and/or additionally, the temperature sensors T1-T9 can be positioned as close as possible to the subject's skin.

In general, the thermal contact between the thermistors T1-T9 and the subject's body improves when the distance between the thermistors T1-T9 and the subject's body is reduced. In some preferred embodiments, the thermal contact between the thermistors T1-T9 in the subject's body is maximized by mounting the thermistors T1-T9 on the front surface of the second flex circuit 60 and/or by positioning a thin layer of conductive adhesive or conductive hydrogel between the subject's skin and the front surface of the second flex circuit 60.

In some preferred embodiments, one or more additional layers of material can be positioned between the thermistors T1-T9, as long as they do not unduly impair the thermal contact between the thermistors T1-T9 and the subject's skin. To this end, any such additional layers should preferably be thin and have high thermal conductivity. Examples of materials that are suitable for such additional layers include a sheet of graphite that is coated (on the skin side) with a conductive adhesive (e.g., a dry conductive carbon adhesive). And as noted above, both graphite and dry conductive carbon adhesive have the additional advantage in that they prevent ions from flowing into or out of the subject's body.

In some alternative embodiments (not shown), instead of providing the entire electrode assembly to the end-user as a single integrated unit, the electrode assembly may be divided into two separate components that are provided to the end-user as a kit. One of the components in the kit includes the electrode elements E1-E9 and a support 50, 80 configured to hold the electrode elements, while the other component in the kit includes the second flex circuit 60 and the temperature sensors T1-T9. To use these embodiments, the end-user can attach the two components to each other and subsequently apply them to his or her body. Alternatively, the end-user can initially apply the temperature-sensing component to his or her body, and subsequently apply the component with the electrode elements directly on top (i.e., to the rear) of the temperature-sensing component. Subsequently, operation of this embodiment will be the same as the embodiment described above in connection with FIGS. 1-4.

Note that the two-part approach described above in connection with FIGS. 2-4 is not mandatory, and the electrode elements E1-E9 and the temperature sensors T1-T9 can all be incorporated into a single unitary subassembly, as long as the temperature sensors T1-T9 are located between the electrode elements E1-E9 and the subject's body. For example, in one such embodiment (not shown), instead of mounting the temperature sensors T1-T9 to a second flex circuit 60, the second flex circuit 60 is omitted, and the temperature sensors T1-T9 are mounted to a mesh of insulated wires positioned in front of the at least one electrode element E1-E9. These insulated wires are configured to route electrical signals to the temperature sensors T1-T9. In these embodiments, it is preferable for the plurality of temperature sensors T1-T9 and the plurality of insulated wires to collectively occupy an area that is less than 10% of a sum of all the areas of all the electrode elements, so that the coupling between the electrode elements E1-E9 and the subject's body is not unduly blocked.

Returning to FIG. 1, each of the electrode assemblies 50, 60, E1-E9, T1-T9 (i.e., the electrode assembly on the left and the electrode assembly on the right) has an associated heat exchanger 70 that is disposed in thermal contact with the at least one electrode element E1-E9. Note that the thermal contact between the heat exchanger 70 and the at least one electrode element E1-E9 need not be a direct thermal coupling between those components. To the contrary, intervening components may be disposed therebetween, as long as the intervening components conduct heat between the heat exchanger 70 and the at least one electrode element E1-E9. The heat exchanger 70 is positioned behind the at least one electrode element E1-E9 and is configured to remove heat from the at least one electrode element. In some embodiments, including the embodiment depicted in FIG. 1, the heat exchanger 70 relies on a flow of a liquid to carry the heat away. This liquid could be, for example, water, oil, etc. In alternative embodiments (not shown), the heat exchanger could be a passive heat sink positioned in contact with something cold (e.g., a cooling blanket, a mass of ice, etc.). In alternative embodiments (not shown), instead of positioning a separate heat exchanger 70 at each of the electrode assemblies, a single heat exchanger can be used to cool both of the electrode assemblies. In other alternative embodiments, the heat exchangers are omitted.

To apply TTFields to the target region in the subject's body using the system depicted in FIG. 1, a first electrode assembly 50, 60, E1-E9, T1-T9 is placed so that at least one first electrode element E1-E9 within the first electrode assembly is positioned against the subject's body, with a plurality of first temperature sensors T1-T9 sandwiched between the at least one first electrode element and the subject's body so that the first temperature sensors are in thermal contact with the subject's body. And a second electrode assembly 50, 60, E1-E9, T1-T9 is placed so that at least one second electrode element E1-E9 within the second electrode assembly is positioned against the subject's body, with a plurality of second temperature sensors T1-T9 sandwiched between the at least one second electrode element and the subject's body so that the second temperature sensors are in thermal contact with the subject's body. Each of the at least one first electrode elements and each of the at least one second electrode elements has a respective rear surface.

A first heat exchanger 70 is positioned in thermal contact with the rear surfaces of the at least one first electrode element E1-E9, and a second heat exchanger 70 is positioned in thermal contact with the rear surfaces of the at least one second electrode element E1-E9. (Note yet again that these thermal contacts can be either direct thermal contact or indirect thermal contact through intervening components.)

Applying an alternating voltage between the at least one first electrode element E1-E9 and the at least one second electrode element E1-E9 will couple an electric field into the subject's body, and this electric field will pass through the target region. This will cause the at least one first electrode element E1-E9 and the at least one second electrode element E1-E9 to heat up above the ambient temperature, and the amount of heating will depend on the current that passes through the at least one first electrode element and the at least one second electrode element.

The first heat exchanger 70 removes heat from the at least one first electrode element E1-E9, and the second heat exchanger 70 removes heat from the at least one second electrode element E1-E9. In the FIG. 1 example, each of these heat exchangers operates by passing a respective cooling fluid through the heat exchanger. But in alternative embodiments, other types of heat exchangers (e.g. passive heat exchangers) can be used.

Because the temperature sensors T1-T9 are in thermal contact with the subject's body, the temperature of the subject's skin in front of the first and second electrode assemblies can be monitored by the first temperature sensors T1-T9 and the second temperature sensors T1-T9 that are built into the first and second electrode assemblies, respectively. This is accomplished by determining a plurality of first temperatures based on outputs of the plurality of first temperature sensors T1-T9, and determining a plurality of second temperatures based on outputs of the plurality of second temperature sensors T1-T9.

The amplitude of the alternating voltage and/or the passing of the first cooling fluid through the first heat exchanger 70 are adjusted based on the determined plurality of first temperatures. And the amplitude of the alternating voltage and/or the passing of the second cooling fluid through the second heat exchanger 70 are adjusted based on the determined plurality of second temperatures. These adjustments are preferably designed so that the current from the AC voltage generator 20 will be as high as possible without raising the temperature of the subject's skin beneath the electrode assemblies above the safety threshold (e.g., 41° C.).

Notably, due to the positioning of the temperature sensors T1-T9 in thermal contact with the subject's body in front of the respective electrode elements E1-E9 in the respective electrode assembly, each of the temperature sensors T1-T9 will more accurately reflect the temperature of the subject's skin (as compared to the prior art approaches where the temperature sensors are not positioned in front of the electrode elements). This is because the thermistors T1-T9 in the FIGS. 1-4 embodiment are in thermal contact with the subject's body and are closer to the subject's skin than the thermistors in the prior art. And this will enable the system to operate at currents that are higher than the prior art systems without running a risk that the temperature of the subject's skin beneath the electrode assemblies will exceed the safety threshold. For example, the prior art Optune® system for applying TTFields to a subject's body always operated below currents of 4 A due to thermal considerations. But when active cooling is combined with temperature sensors that are positioned in thermal contact with the subject's body and in front of the electrode elements, as described herein, the system can achieve significantly higher currents (e.g., >5 A, >6 A, >8 A, >10 A, >12 A, or >15 A). And these higher operating currents will yield higher efficacy of treatment.

Finally, while the embodiment depicted in FIGS. 1-4 is illustrated and described above with nine electrode elements E1-E9 and nine temperature sensors T1-T9, the number of electrode elements and the number of temperature sensors can vary (e.g. from 1 to 50). Similarly, while FIG. 1 depicts two electrode assemblies (i.e., one of the left and one on the right of the target region), more than two electrode assemblies may be used (e.g., 3, 4, or more than 4).

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for applying an alternating electric field to a subject's body, the apparatus comprising:
at least one electrode element, wherein each electrode element has a respective front face that has a respective area;
a support configured to hold the at least one electrode element adjacent to the subject's body, so that the front faces of the at least one electrode element face the subject's body; and
a plurality of temperature sensors positioned in front of the at least one electrode element so that when the support holds the at least one electrode element adjacent to the subject's body, the plurality of temperature sensors will be situated between the front faces of the at least one electrode element and the subject's body, in thermal contact with the subject's body.

2. The apparatus of claim 1, wherein each of the temperature sensors comprises a thermistor.

3. The apparatus of claim 1, further comprising a plurality of insulated wires positioned in front of the at least one electrode element, wherein the plurality of insulated wires is configured to route electrical signals to the temperature sensors.

4. The apparatus of claim 3, wherein the plurality of temperature sensors and the plurality of insulated wires collectively occupy an area that is less than 10% of a sum of all the areas of all the electrode elements.

5. The apparatus of claim 1, further comprising a flex circuit configured to support the plurality of temperature sensors and to provide an electrical interface with the plurality of temperature sensors.

6. The apparatus of claim 5, wherein the flex circuit has an insulating substrate and a plurality of conductive traces, wherein the flex circuit is oriented so that the insulating substrate is in front of the plurality of conductive traces, and
wherein the insulating substrate is shaped and dimensioned to prevent the plurality of conductive traces from making contact with the subject's body.

7. The apparatus of claim 1, wherein the support is positioned behind the at least one electrode element.

8. The apparatus of claim 1, further comprising a heat exchanger disposed in thermal contact with the at least one electrode element, wherein the heat exchanger is positioned behind the at least one electrode element and is configured to remove heat from the at least one electrode element.

9. The apparatus of claim 8, wherein the heat exchanger relies on a flow of a liquid to carry the heat away.

10. The apparatus of claim 1, further comprising a layer of insulating material having a dielectric constant greater than 10 disposed on the front faces of the at least one electrode element,
wherein the plurality of temperature sensors are positioned in front of the layer of insulating material.

11. The apparatus of claim 1, further comprising:
a layer of insulating material having a dielectric constant greater than 10 disposed on the front faces of the at least one electrode element, wherein the layer of insulating material has a front face; and
a sheet of graphite disposed on the front face of the layer of insulating material,
wherein the plurality of temperature sensors are positioned in front of the layer of graphite.

12. The apparatus of claim 1, further comprising:
a sheet of graphite disposed in front of the plurality of temperature sensors so that when the support holds the at least one electrode element adjacent to the subject's body, the sheet of graphite will be situated between the plurality of temperature sensors and the subject's body; and
a layer of conductive adhesive disposed in front of the sheet of graphite so that when the support holds the at least one electrode element adjacent to the subject's body, the layer of conductive adhesive will be situated between the sheet of graphite and the subject's body.

13. The apparatus of claim 1, further comprising a flex circuit having an insulating substrate and a plurality of conductive traces,
wherein the at least one electrode element comprises a plurality of electrode elements,
wherein the flex circuit is positioned so that when the support holds the plurality of electrode elements adjacent to the subject's body, the flex circuit will be situated between the front faces of the plurality of electrode elements and the subject's body,
wherein the plurality of temperature sensors are affixed to the flex circuit, and
wherein the plurality of conductive traces of the flex circuit are configured to provide an electrical interface with the plurality of temperature sensors.

14. The apparatus of claim 13, wherein each of the temperature sensors comprises a thermistor.

15. The apparatus of claim 13, wherein the flex circuit is shaped and dimensioned to cover less than 10% of a sum of all the areas of all the electrode elements.

16. The apparatus of claim 13, wherein a sum of all the areas of all the electrode elements is at least 10 cm².

17. A method for applying an alternating electric field to a subject's body, the method comprising:
positioning at least one first electrode element against the subject's body, with a plurality of first temperature sensors sandwiched between the at least one first electrode element and the subject's body so that the first temperature sensors are in thermal contact with the subject's body, wherein each of the at least one first electrode elements has a respective rear surface;
positioning a first heat exchanger in thermal contact with the rear surfaces of the at least one first electrode element;
positioning at least one second electrode element against the subject's body, with a plurality of second temperature sensors sandwiched between the at least one second electrode element and the subject's body so that the second temperature sensors are in thermal contact with the subject's body, wherein each of the at least one second electrode elements has a respective rear surface;
positioning a second heat exchanger in thermal contact with the rear surfaces of the at least one second electrode element;
applying an alternating voltage between the at least one first electrode element and the at least one second electrode element;
passing a first cooling fluid through the first heat exchanger to remove heat from the at least one first electrode element;
passing a second cooling fluid through the second heat exchanger to remove heat from the at least one second electrode element;
determining a plurality of first temperatures based on outputs of the plurality of first temperature sensors;
determining a plurality of second temperatures based on outputs of the plurality of second temperature sensors;
adjusting at least one of (a) an amplitude of the alternating voltage and (b) the passing of the first cooling fluid through the first heat exchanger based on the determined plurality of first temperatures; and
adjusting at least one of (i) an amplitude of the alternating voltage and (ii) the passing of the second cooling fluid through the second heat exchanger based on the determined plurality of second temperatures.

18. The method of claim 17, wherein the at least one first electrode element comprises a plurality of first electrode elements, and wherein each of the plurality of first temperature sensors is sandwiched between a respective one of the plurality of first electrode elements and the subject's body, and
wherein the at least one second electrode element comprises a plurality of second electrode elements, and wherein each of the plurality of second temperature sensors is sandwiched between a respective one of the plurality of second electrode elements and the subject's body.

19. The method of claim 17, wherein each of the first temperature sensors and each of the second temperature sensors comprises a thermistor.

20. A kit for applying an alternating electric field to a subject's body, the kit comprising:
a first component that includes
a plurality of electrode elements, wherein each of the electrode elements has a respective front face that has a respective area, and
a support configured to hold the plurality of electrode elements adjacent to the subject's body, so that the front faces of the plurality of electrode elements face the subject's body; and
a second component that includes
a flex circuit having an insulating substrate and a plurality of conductive traces, and
a plurality of temperature sensors affixed to the flex circuit,
wherein the plurality of conductive traces of the flex circuit are configured to provide an electrical interface with the plurality of temperature sensors, and
wherein the flex circuit is configured to fit between the front faces of the plurality of electrode elements and the subject's body when the support holds the plurality of electrode elements adjacent to the subject's body.
